(19) United States
(12) Patent Application Publication
(10) Pub. No.: US 2007/0073102 A1
(43) Pub. Date: Mar. 29, 2007

(54) Endoscope apparatus

(76) Inventors: Kiyotaka Matsuno, Sagamihara-shi (JP); Takumi Dejima, Tokyo (JP); Ryo Minosawa, Tsukui-gun (JP); Manabu Miyamoto, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/237,487

(22) Filed: Sep. 27, 2005

(51) Int. Cl. A61B 1/00; A61B 1/04

(52) U.S. Cl. 600/102; 600/114; 600/130

(57) ABSTRACT

The endoscope apparatus comprises an endoscope which has an imaging element and a monitor which displays an image imaged by the imaging element. The endoscope is provided with an internal insertion portion which is movably arrangeable inside a body cavity, and an operation unit for changing the position of the internal insertion portion, which is arrangeable outside the body. The internal insertion portion and the operation unit are arranged on a common axis so as to interpose a body wall therebetween.

### BACKGROUND OF THE INVENTION

1. Field of the Invention

The present invention relates to an endoscope apparatus. In detail, the present invention relates to an endoscope apparatus which moves a treating unit or an observation unit which has an imaging element and which is movably arrangeable inside of a body cavity, using an operation unit which is arranged outside of the body so as to interpose a body wall with the treating unit or the observation unit.

2. Description of the Related Art

Conventionally, in surgery using an endoscope, a plurality of openings whose diameter is 3-10 mm are made in a body wall, and multiple forceps and a rigid endoscope are inserted into the body cavity through the openings. Then, various operations are performed using the forceps and the endoscope.

As a rigid endoscope used in such surgery, reference is made, for example to the device disclosed in Japanese Unexamined Patent Application, First Publication No. H11-326783. The rigid endoscope comprises a rigid insertion tube and a monitor connected to a base end of the rigid insertion tube. In the rigid insertion tube, an object lens system, an imaging element and a signal cable are disposed successively from a tip of the rigid insertion tube. The monitor is electrically connected to the imaging element through the signal cable. With the rigid endoscope, the tip of the rigid insertion tube is inserted through the opening made in the body wall, and the image of the inside of the body cavity captured by the imaging element is displayed on the monitor.

In addition, as an endoscope used in surgery, in Japanese Unexamined Patent Application, First Publication No. 2003-1117220 reference is made to an apparatus provided with an internal robot and a 3-D gradient magnetic field generator. With this apparatus, the internal robot is inserted into the body cavity, and the internal robot is moved by remote control using the magnetism produced by the 3-D gradient magnetism generator. Then, the images of the inside of the body cavity are captured by the imaging element attached in the internal robot, and the images are transmitted to outside of the body using a transmitting device built in the internal robot.

### SUMMARY OF THE INVENTION

The present invention is an endoscope apparatus including: an endoscope which has an imaging element; and a monitor which displays an image imaged by the imaging element; wherein the endoscope is provided with an internal insertion portion which is movably arrangeable inside a body cavity, and an operation unit for changing the position of the internal insertion portion, which is arrangeable outside the body, and wherein the internal insertion portion and the operation unit are arranged on a common axis so as to interpose a body wall therebetween.

### BRIEF DESCRIPTION OF THE DRAWINGS

FIG. 1 is a schematic view of an endoscope apparatus relating to the first embodiment of the present invention.

FIG. 2 is an enlarged sectional view of a part showing the state of use of the endoscope apparatus relating to the first embodiment of the present invention.

FIG. 3 is a schematic plan view showing the endoscope apparatus relating to the second embodiment of the present invention.

FIG. 4 is a sectional view taken along a line A-A of FIG. 3, showing the endoscope apparatus relating to the second embodiment of the present invention.

FIG. 5 is an enlarged sectional view of a part of FIG. 4, showing the endoscope apparatus relating to the second embodiment of the present invention.

FIG. 6 is a sectional view taken along a line B-B of FIG. 5 showing the endoscope apparatus relating to the second embodiment of the present application.

FIG. 7 is a view taken along an arrow C of FIG. 3 showing the endoscope apparatus relating to the second embodiment of the present invention.

FIG. 8 is a sectional view taken along a line D-D of FIG. 7 showing the endoscope apparatus relating to the second embodiment of the present invention.

FIG. 9 is an enlarged view of a part E indicated in FIG. 3, showing the endoscope apparatus relating to the second embodiment of the present invention.

FIG. 10 is a schematic view of the endoscope apparatus relating to the third embodiment of the present invention.

FIG. 11 is a sectional view, showing the state of use of the endoscope apparatus relating to the third embodiment of the present invention.

FIG. 12 is a schematic view of the endoscope apparatus relating to the fourth embodiment of the present invention.

### DESCRIPTION OF THE PREFERRED EMBODIMENTS

Preferred embodiments of the present invention will be explained hereafter, with reference to the drawings. In all of the drawings, even in different embodiments, the same or corresponding components are labeled the same, and duplicate explanations are omitted.

### First Embodiment

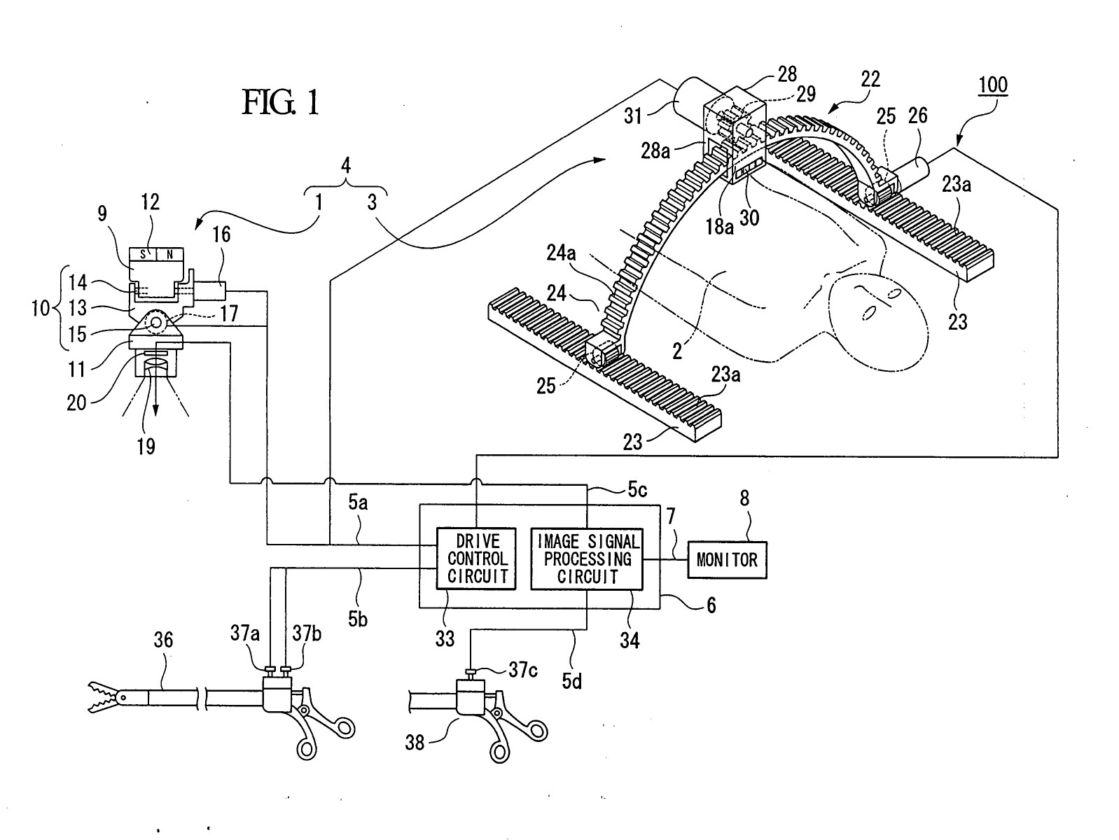

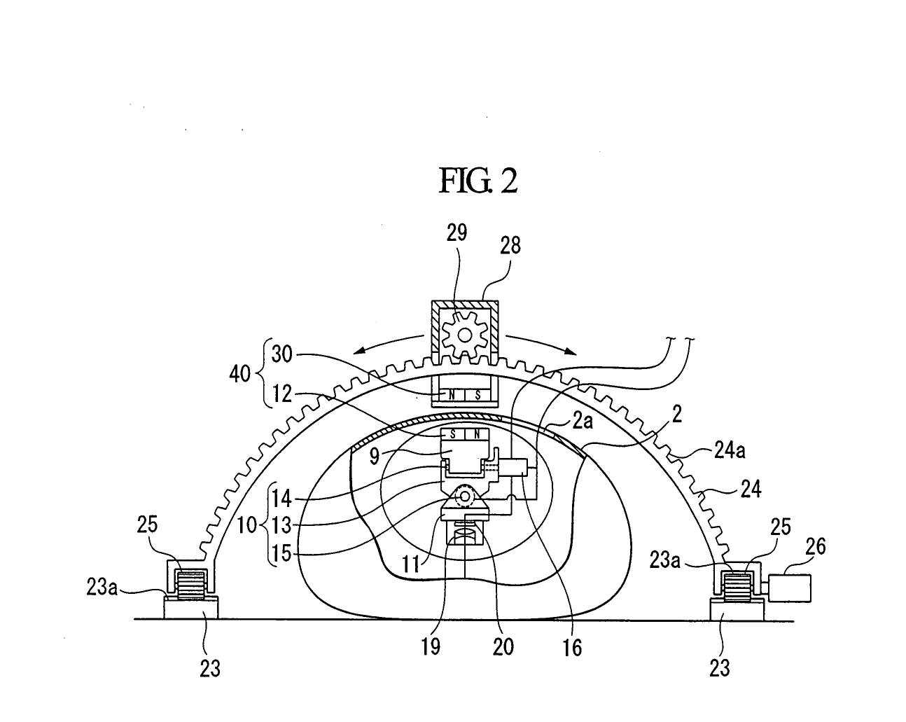

An explanation of an endoscope apparatus relating to the first embodiment of the present invention is described hereafter, with reference to FIG. 1 and FIG. 2.

FIG. 1 is a schematic view of the endoscope apparatus of the first embodiment of the present invention. An endoscope apparatus 100 includes an endoscope 4; a processor which is electrically connected to the endoscope 4 through transmission wires 5a and 5c, and a monitor 8 which is electrically connected to a processor 6 through a transmission wire 7 and which displays an image captured by the endoscope 4. The endoscope 4 includes an internal insertion portion 1 which is movably arranged inside of the body cavity, and an operation unit 3 which is movably arranged outside of the body so as to interpose a body wall 2 with the internal insertion portion 1, and which changes the position of the internal insertion portion 1.

The internal insertion portion 1 includes a base portion 9, an observation unit main body 11 which is attached to the base portion 9 through a joint portion 10 so that the angle of the observation unit main body 11 relative to the base portion 9 is variable. A magnet 12 is attached to a body wall side end portion of the internal insertion portion 1 arranged inside of the body cavity. For example, a rectangular permanent magnet is applied as the magnet 12.

The joint portion 10 includes: an intermediate body 13 which is disposed between the base portion 9 and the observation unit main body 11; a rotation shaft 14 which is attached to the base portion 9 and the intermediate body 13; and a rotation shaft 15 which is attached to the intermediate body 13 and the observation unit main body 11. The rotation shaft 14 and the rotation shaft 15 are arranged so as to mutually cross. The rotation shaft 14 is connected to a motor 16, and the rotation shaft 15 is connected to a motor 17. The rotation shaft 14 is rotated in both forward and reverse directions by the motor 16, the rotation shaft 15 is rotated in both forward and reverse directions by the motor 17.

The observation unit main body 11 includes an object optical system 19 and an imaging element 20.

The operation unit 3 is moved along the body wall 2 by being guided by a guide portion 22, and is fixed on a predetermined position. The guide portion 22 includes two rectilinear rails 23 which are disposed parallel to each other, and an arched rail 24 which is disposed between the two rectilinear rails 23 and which is movable along the two rectilinear rails 23. The arched rail 24 movably guides the operation unit 3 along an arched path thereof.

Pinions 25 are attached to both ends of the arched rail 24, and engage racks 23a are formed on upper surfaces of the two rectilinear rails 23. The arched rail 24 moves along the rectilinear rails 23 while of crossing the rectilinear rails 23 by synchronously rotating both the pinions 25 by a motor 26. Note that, FIG. 1 shows only the connection of the motor 26 to one of the pinions 25; a different motor is connected to the other pinion 25.

The operation unit 3 includes: a housing 28; a pinion 29 which is rotatably supported by the housing 28, and which engages with a teeth portion 24a formed on an upper surface of the arched rail 24; and a magnet 30 which is attached to a lower part of the housing 28. The housing 28 is provided with side portions 28a which interpose both sides of the rectilinear rails 23.

There is a coupler 40 which moves the internal insertion portion 1 following the movement of the operation unit 3 inside the body cavity so that the internal insertion portion 1 and the operation unit 3 are arranged on an axis interposing the body wall 2 while the operation unit 3 is guided along the body wall 2 by the guide portion 22. The coupler 40 includes the magnet 12 which is disposed on the operation unit 3, and the magnet 30 which is disposed on the internal insertion portion 1. (Refer to FIG. 2)

In addition, the magnet 30 of the operation portion 3 has a magnetic force for raising up the internal insertion portion 1 toward the body wall 2 through the magnet 12 interposing the body wall 2. The magnet 30 may be an electromagnet or a superconductive magnet. The same applies to the magnet 12 belonging to the base 9.

The magnet 30 of the operation unit 3 and the magnet 12 of the internal insertion portion 1 are positioned so that the rotation shaft 14 is parallel to a surface of the arched rail 24, and the rotation shaft 15 crosses the surface of the arched rail 24 when the magnet 30 of the operation unit 3 absorbs the magnet 12 of the internal insertion portion 1 in a regular state interposing the body wall 2.

Note that, in these figures exemplified above, by assembling the pinions 25 and the engage rack 23a, the arched rail 24 is moved relative to the rectilinear rails 23. However, the invention is not limited to this, and through the mechanism of a ball screw, the arched rail 24 may also be moved relative to the rectilinear rails 23. In addition, when the operating unit 3 is moved along the arched rail 24, similar use may be made of the mechanism of the ball screw.

The processor 6 includes a drive control circuit 33 and an image signal processing circuit 34.

The drive control circuit 33 is electrically connected to the motors 16, 17, 26, and a motor 31 through the transmission wire 5a, and is electrically connected to switches (inputting unit) 37a, 37b of rigid forceps 36 through a transmission wire 5b. The image signal processing circuit 34 is electrically connected to the imaging element 20 through the transmission wire 5c, and is electrically connected to a zoom switch (inputting unit) 37c of rigid forceps 38 through a transmission wire 5d. In addition, the image signal processing circuit 34 is electrically connected to the monitor 8 through the transmission wire 7.

The switch 37a is capable of four-directional inputting as a joystick or the like. For example, when the switch 37a is operated in a lateral direction, the drive control circuit 33 performs a movement of the operation unit 3 along the arched rail 24 by driving a motor 31, when the switch 37a is operated in a back and forth direction, the drive control circuit 33 performs a movement of the arched rail 24 along the rectilinear rail 23 by driving the motor 26. The switch 37b is also capable of four-directional inputting as a joystick or the like. For example, when the switch 37b is operated in a lateral direction, the drive control circuit 33 performs a rotation of the intermediate body 13 relative to the base portion 9 by driving the motor 16, and when the switch 37b is operated in a back and forth direction, the drive control circuit 33 performs a rotation of the observation unit main body 11 relative to the intermediate body 13 by driving the motor 17.

In addition, the zoom switch 37c is used for controlling image zooming by operating the zoom switch 37c, and enlargement and reduction of the picture image is performed through the image signal processing circuit 34.

Next, an explanation of the operation of the above composed endoscope apparatus 100 is provided.

As shown in FIG. 1, the arched rail 24 and the rectilinear rails 23 are disposed along the body wall 2 of a patient.

Next, the internal insertion portion 1 is inserted into the body cavity through the opening in the body wall 2, and is brought close to the pre-fixed operating unit 3 of the arched rail 24. Then, by applying an absorption force between the magnet 30 of the operation unit 3 and the magnet 12 of the internal insertion portion 1, the internal insertion portion 1 is fixed to an inner surface of the body wall 2 in a state of pressing the internal insertion portion 1 to the inside of the body wall 2.

The information obtained by the observation unit main body 11 of the internal insertion portion 1 is projected on the monitor 8 through the image signal processing circuit 34. An operator treats the patient by operating the rigid forceps 36, 38, which have been inserted through a different opening 2a in the body wall 2 while viewing the monitor 8.

When viewing the patient from the side surface, the movement of the operation unit 3 along the arched rail 24, and the movement of the arched rail 24 along the rectilinear rails 23 are controlled by operating the switch 37a like a joystick which is attached to the rigid forceps 36, and the operation unit 3 is moved along the body wall 2. At this time, the internal insertion portion 1 is moved along the inside of the body wall 2 magnetically by the internal insertion portion 1 and the operation unit 3. Further, when the switch 37b is operated, the motors 16 and 17 are driven, and the inclination angle of the observation unit main body 11 relative to the base 9 is adjusted.

Therefore, the operator can treat a target area while observing the target area from a desired angle.

Similarly, the operator can treat the target area while he is viewing the image at a desired size using the monitor 8 through the image signal processing circuit 34 by operating the zoom switch 37c attached to the rigid forceps 38.

According to the endoscope apparatus as describe above, the internal insertion portion 1 and the operation unit 3 are magnetically attached to each other so as to interpose the body wall 2 therebetween, and the operation unit 3 is moved along the body wall 2 outside the body, then the internal insertion portion 1 is moved following the operation unit 3. Therefore, it is possible to move the internal insertion portion 1 relative to the body wall 2 without restriction of the position of the opening 2a made in the body wall 2, and it is possible to observe the target area from any direction.

In addition, since the internal insertion portion 1 is attached to the operation unit 3 so that the body wall 2 is interposed between the internal insertion portion 1 and the operation unit 3, the position of the operation unit 3 is roughly equal with the position of the internal insertion portion 1 (that is the position of the observation unit main body 11). Therefore, it is easy to intuitively understand the position from which the image displayed on the monitor 8 is captured, and it is easy to perform orientation. As a result, it is possible to treat the target area appropriately.

Further, according to the endoscope apparatus as described above, since it is not necessary to use a large-size magnetic field generator, it is possible to reduce the space and number of components required, thereby achieving low-cost.

In conventional surgery using an endoscope apparatus, an operator who operates a rigid endoscope known as a “scopist”, and another operator who operates two rigid forceps are required. However, according to the endoscope apparatus as described above, an operator who operates the two rigid forceps 36, 38 is able to move the internal insertion portion 1 and to operate zooming. Therefore, only one operator is required to operate the forceps 36, 38 and the endoscope, and the above mentioned “scopist” is not required.

### Second Embodiment

An explanation of the endoscope apparatus relating to the second embodiment of the present invention is described hereafter.

This second embodiment differs from the above-mentioned first embodiment of the present invention as follows. That is, while the operation unit 3 is guided along the body wall 2 by the guide portion 22, a coupler 40 moves the internal insertion portion 1 following the movement of the operation unit 3 so that the internal insertion portion 1 and the operation unit 3 are arranged on a common axis so as to interpose the body wall 2 therebetween.

In the first embodiment, the operation unit 3 is moved by the motors 26, 31. However, in the second embodiment, the operation unit 3 is moved manually without the driving force of the motor or the like. Therefore, the endoscope apparatus of the second embodiment does not include the motors 26, 31. In this point as well, the second embodiment differs from the first embodiment.

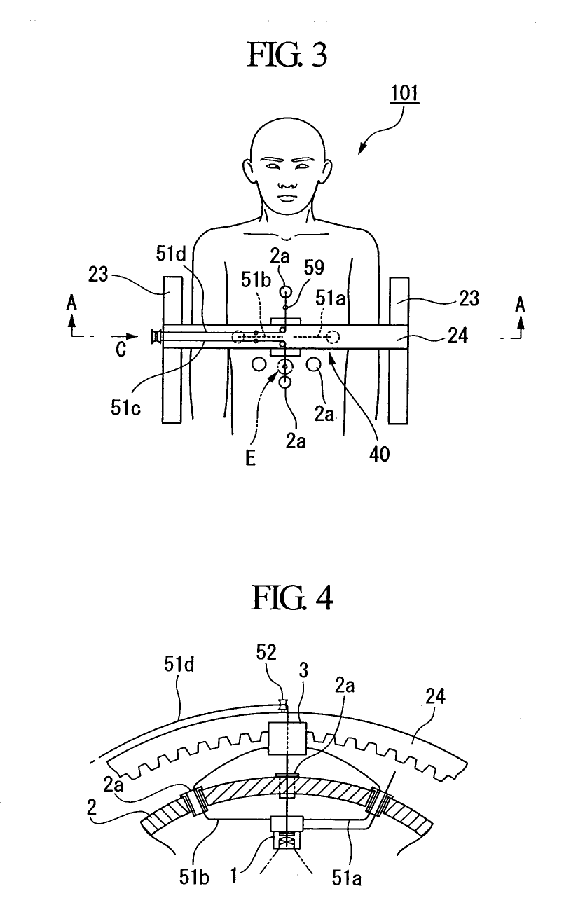

An explanation of the endoscope apparatus relating to the second embodiment is described hereafter, with reference to FIG. 3 to FIG. 9.

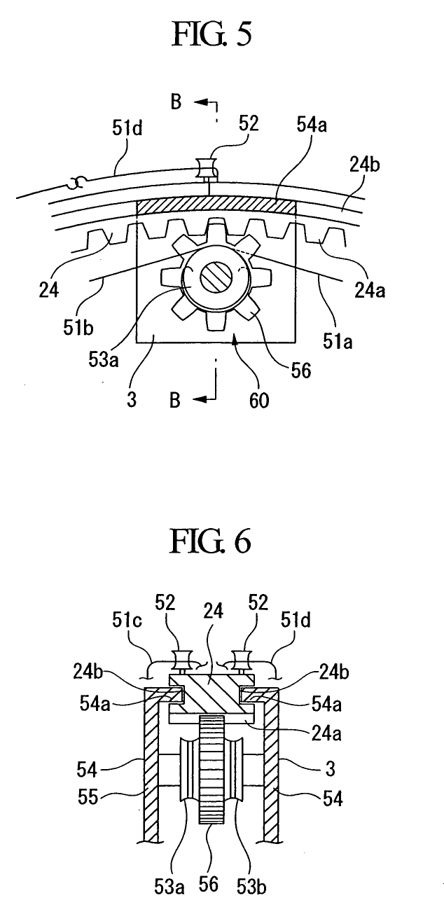

FIG. 3 is a schematic plan view showing an endoscope apparatus 101. FIG. 4 is a sectional view taken along a line A-A of FIG. 3, FIG. 5 to FIG. 9 are enlarged sectional views of an essential part of the endoscope apparatus 101.

The internal insertion portion 1 is connected to the operation unit 3 disposed outside of the body or the guide portion 22 guiding the operation unit 3 through a number of ropes (elongated body) 51a, . . . which have flexibility and elasticity like rubber. This second embodiment describes an example in which there are four ropes 51.

One end of each of the four ropes 51a, . . . is connected to the base portion 9 of the internal insertion portion 1 which is disposed in the body cavity. The other ends of the two ropes 51a, 51b among the four ropes 51a, . . . extend from the internal insertion portion 1 in the lateral direction in the body cavity, and further extend to the outside of the body through the opening 2a made in the body wall 2. Then, the other ends of the two ropes 51a, 51b are turned back, and are connected to the operation unit 3. The remaining other ends of the two ropes 51c, 51d among the four ropes 51a, . . . extend in the back and forth direction so as to become parallel to the rectilinear rails 23 in the body cavity, and further respectively extend to the outside of the body through the opening 2a made in the body wall 2. Then, the other ends of the two ropes 51c, 51d are turned back, and are connected to one end of the arched rail 24 via pulleys 52 disposed on the top of the arched rail 24.

As shown in FIG. 5 and FIG. 6, the operation unit 3 is provided with pulleys 53a, 53b for winding up or reeling out the ropes 51a, 51b, respectively. The other ends of the ropes 51a, 51b are fixed on the pulleys 53a, 53b respectively, and the other end of the rope 51a is wound around the pulleys 53a in the opposite direction to the other end of the rope 51b wound around the pulley 53b. When the operation unit 3 is positioned in the center of the arched rail 24, the length of the ropes 51a, 51b wound around the pulleys 53a, 53b is approximately half the length of the arched rail 24.

The operation unit 3 is movable along an inner surface of the arched rail 24 by inserting upper end engaging parts 54a formed on both side portions 54 into arched grooves 24b formed on a side part of the arched rail 24. A pinion 56 is supported by a shaft 55, and the pinion 56 is engaged on the teeth portion 24a formed on the inside of the arched rail 24. The pulleys 53a, 53b are attached to both ends of the pinion 56 so that the pulleys 53a, 53b rotate as a unit with the pinion 56. Thus, the pulleys 53a, 53b are rotated when the operation unit 3 is moved along the arched rail 24.

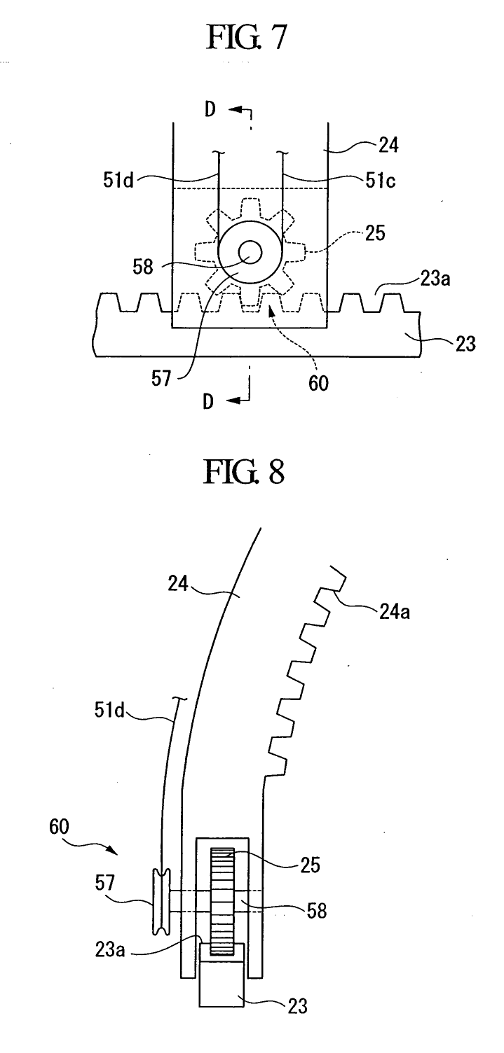

As shown in FIG. 7 and FIG. 8, a pulley 57 for winding up or reeling out the ropes 51a, 51b is disposed on an end of the arched rail 24. The other ends of the ropes 51c, 51d are fixed on the pulley 57, and the other end of the rope 51c is wound the pulley 57 in the opposite direction to the other end of the rope 51d wound around the pulley 57. When the operation unit 3 and the arched rail 24 are positioned in the center of the rectilinear rails 23, the length of the ropes 51c, 51d wound around the pulley 57 is approximately half the length of the rectilinear rails 23.

The pinion 25 which is engaged on the engage rack 23a of the rectilinear rail 23 is supported on a lower end of the arched rail 24 through a shaft 58, and the pulley 57 is attached to the shaft 58 so as to rotate as a unit with the pinion 25. Thus, the pulley 57 is rotated in a predetermined direction, when the arched rail 24 is moved along the rectilinear rails 23.

Note that, the pulley 57 is not restricted to just one, but two pulleys may be attached to the shaft 58 respectively corresponding to the ropes 51c, 51d. In this case, the pulleys are attached so as to rotate as a unit with the pinion 25.

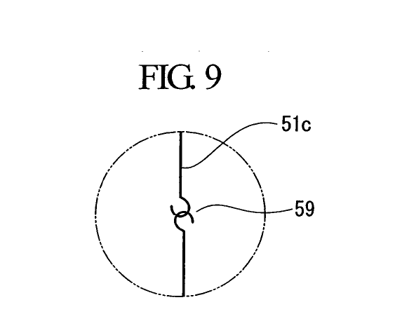

In addition, as shown in FIG. 9, the four ropes 51a, . . . each have hook-like connecting portions 59. The connecting portions 59 are detachable so as to be able to divide the rope at the mid-point.

In the second embodiment, the pulleys 23a, 23b, 57 and the pinions 25, 56 compose a length of rope adjusting portion 60 (length of elongated body adjusting portion) which adjusts the length of the ropes 51a, 51b, 51c, 51d. Further, the length of rope adjusting portion 60 and the ropes 51a, 51b, 51c, 51d compose the coupler 40 which moves the internal insertion portion 1 following the movement of the operation unit.

Next, an explanation of the operation of the above composed endoscope apparatus 101 is provided.

The internal insertion portion 1 is inserted into the body cavity through one of the four openings 2a made in the body wall 2 while remaining the rope 51a outside the body. At this time, each of connecting portions 59 of the three ropes 51b, 51c, 51d which are inserted into the body cavity, is detached.

Next, ends of the three ropes 51b, 51c, 51d are removed from the body through the remaining three opening 2a, using rigid forceps or the like. The ends of the removed ropes 51b, 51c, 51d are attached to the other ends of the ropes 51b, 51c, 51d remaining in the operation unit 3 and the guide portion 22 through the connecting portions 59.

When the operation unit 3 is moved along the arched rail 24 manually, the pinion 56 which engages with the teeth portion 24a of the arched rail 24 is rotated, and the pulleys 53a, 53b are also rotated. According to this rotation, the rope 51a is wound up by the pulley 53a, and the rope 51b is reeled out from the pulley 53b. Therefore, the internal insertion portion 1 is pulled by the rope 51c wound up, and is moved in the body cavity coupling the operation unit 3.

When both movements are combined, since the ropes 51a, 51b, 51c, 51d, which have elasticity, are extended with a certain amount of the length, the length of the rope which is required is compensated.

Other operations are the same as those of the first embodiment.

According to the endoscope apparatus 101 as describe above, the internal insertion portion 1 and the operation unit 3 are coupled by the ropes 51a . . . so as to interpose the body wall 2 therebetween, and the operation unit 3 is moved along the body wall 2 outside the body, then the internal insertion portion 1 is moved following the operation unit 3. Therefore, it is possible to move the internal insertion portion 1 relative to the body wall 2 without restriction of the position of the opening 2a made in the body wall 2, and it is possible to observe the target area from any direction. Further, since the internal insertion portion 1 is coupled with the operation unit 3 so that the body wall 2 is interposed between the internal insertion portion 1 and the operation unit 3, it is easy to intuitively understand the position from which the image displayed on the monitor 8 is captured, and it is easy to perform orientation. As a result, it is possible to treat the target area appropriately. Those advantages are the same as those of the first embodiment.

In the second embodiment, since the operation unit 3 is manually moved, and the motor is not used, the overall mechanism can be simplified, keeping costs low.

Note that, in the second embodiment, the operation unit 3 is manually moved. However, the operation unit 3 may be moved by using the driving force of the motor. Further, the endoscope apparatus may be provided with a zooming mechanism for enlarging the image of the imaging element 20 of the internal insertion portion 1, and a joint portion so as to perform angle adjustment of the observation portion main body relative to the base portion.

### Third Embodiment

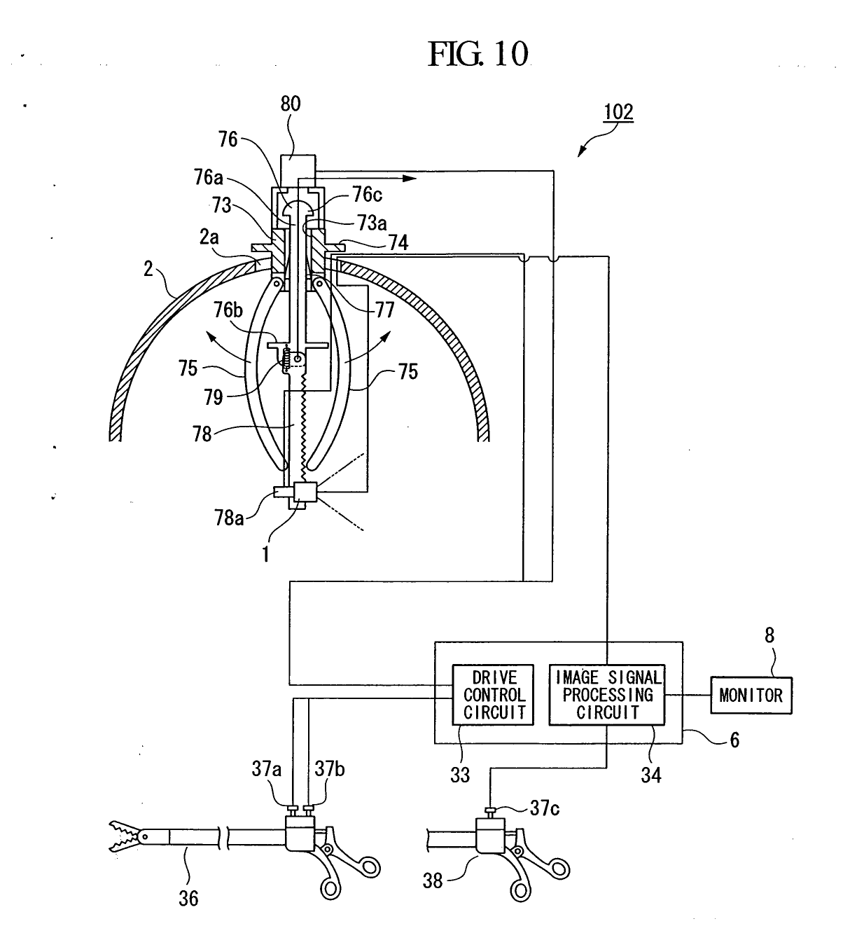

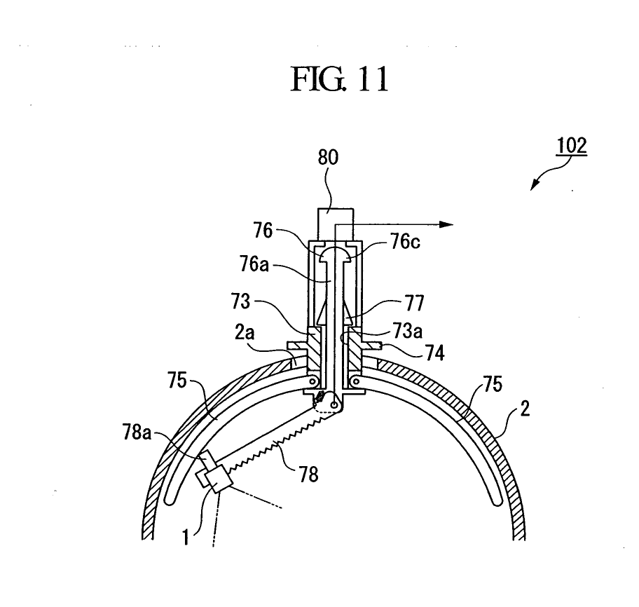

An explanation of the endoscope apparatus relating to the third embodiment of the present invention is described hereafter, with reference to FIG. 10 and FIG. 11.

FIG. 10 is a view showing a state where a part of the endoscope apparatus is inserted into the body cavity, FIG. 11 is a view showing a state where the part of the endoscope apparatus has been inserted into the body cavity.

This third embodiment differs from the above-mentioned first embodiment as follows. That is, there is an internal rail 78, and a bone members 75 for moving the internal rail 78 smoothly in the third embodiment. As shown in those figures, there is the operation unit 3 outside the body in the third embodiment, as in the first and second embodiment. This point is omitted hereunder.

In the third embodiment, the numeral 73 indicates a cylindrical fixing member. A lower end thereof is inserted into the body cavity through the opening 2a made in the body wall 2 and is fixed. A flange portion 74 is formed on the operator's side of the fixing member 73. The diameter of the flange 74 is larger than that of the opening 2a made in the body wall 2. Thus, the insertion of the lower end of the fixing member 73 into the body cavity by more than a certain length is restricted.

A number of (for example, four or eight) the bone members 75 are attached to the lower end of the fixing member 73 so that those bone members 75 are arranged in the circumferential direction in a spaced manner, and are swingably arranged in a plane including a center axis of the fixing member 73. It is preferable that the bone member 75 is gently curved like the internal shape of the body wall 2.

A center rod 76 is inserted into a center hole 73a made in the fixing member 73. The center rod 76 is substantially rod-shaped except that both ends have a small diameter part 76a so as to be able to insert into the center hole 73a of the fixing member 73. Further, a large diameter part 76b is formed on the tip side (lower end side) of the center rod 76, and a handle 76c is formed on the operator's side (upper end side) of the center rod 76.

A shuttlecock portion 77 is disposed on the operator's side of the center rod 76. The shuttlecock portion 77 is spreadable by its own elasticity. When the shuttlecock portion 77 is closed, the center rod 76 can be put through the center hole 73a, when the shuttlecock portion 77 is spread, the center rod can not be put through the center hole 73a, because the shuttlecock portion 77 is spread larger than the diameter of the center hole 73a. Usually, the shuttlecock portion 77 is spread due to its own elasticity. The large diameter part 76b is larger than the diameter of the center hole 73a, and the diameter of the large diameter part 76b is substantially equal to that of a pin support portion of the fixing member 73, which swingably supports the bone members 75.

In addition, the length from the shuttlecock portion 77 to the large diameter part 76b is set to be approximately the same as the length of the center opening 73a of the fixing member 73.

An internal rail 78 is rotatably attached to a lower end of the center rod 76. The internal rail 78 is urged in a direction so that the internal rail 78 is bent relative to the center rod 78, by a spring 79 arranged between the internal rail 78 and the large diameter part 76b.

The internal insertion portion 1 is attached to the internal rail 78 so as to be able to move in a longitudinal direction of the internal rail 78. By a mechanism which is disposed between the internal rail 78 and the internal insertion portion 1 (for example, a mechanism formed by a rack and a pinion), the internal insertion portion 1 can be moved to an optional position, and can be fixed to the optional position. The internal insertion portion 1 is moved by, for example, a motor 78a which is built in the internal insertion portion 1. That is, the internal insertion portion 1 forms an internal guide portion which guides the internal insertion portion 1 in the body cavity. Further, a motor 80 which rotates the center rod 76 is disposed between the fixing member 73 and the center rod 76.

Next, an explanation of the operation of the above composed endoscope apparatus 102 is provided.

The shuttlecock portion 77 is closed manually, and the center rod 76 is pushed into the center hole 73a of the fixing member 73. Further, while the bone members 75 are closed, the fixing member 73 is inserted into the body cavity through the opening 2a made in the body wall 2, until the flange portion 74 contacts the body wall 2.

Next, while the fixing member 73 is held manually, the center rod 76 is pulled up relative to the fixing member 73 until the shuttlecock portion 77 is exposed from the center hole 73a, and the large diameter part 76b contacts the fixing member 73. Accordingly, the fixing member 73 is interposed between the shuttlecock portion 77 and the large diameter part 76b by the shuttlecock portion 77 being spread by the elasticity thereof. As the result, the center rod 76 is fixed to the fixing member 73. Further, by the larger diameter part 76b being pulled up, a base portions of the bone members 75, then the bone members 75 are spread. Furthermore, the internal rail 78 is turned relative to the center rod 76 by a retracting force of the spring 79. In this state, it is possible to observe inside the body cavity.

At this time, the internal insertion portion 1 and operation unit (not shown) are positioned opposite each other so as to interpose the body wall 2 therewith.

While a picture image which is captured by the imaging element of the internal insertion portion 1 is viewed using the monitor 8, the rigid forceps are inserted through the opening 2a which differs from the above-mentioned opening 2a, then the target area is treated. When it is required to observe the target area from a different angle, similar to the first embodiment, the operation unit (not shown) is moved by the switches 37a, 37b which are attached to the rigid forceps 36. The position of the operation unit is detected by a sensor (not shown), and a detection signal of the sensor is sent to the processor 6. A driving signal is sent from the drive control circuit 33 based on the positional signal of the operation unit. The motor 80 is driven by the driving signal, and rotates the center rod 76 until the internal insertion portion 1 corresponds to the operation unit. After that, the motor 78a is driven by the driving signal sent from the drive control circuit 33, thus the internal insertion portion 1 is moved relative to the internal rail 78. Therefore, it is possible to arrange the internal insertion portion 1 disposed inside of the body cavity on the position corresponding to the operation unit (not shown) so as to interpose a body wall 2 therewith.

Also after that, as the need arises, the zoom switch 37c disposed on the rigid forceps 38 is operated, and the target area is observed in an enlarged view.

When the treatment is finished, the motor 78a is driven by the driving signal from the drive control circuit 33, thus the internal insertion portion 1 is moved to the distal end of the internal rail 78. After that, the shuttlecock portion 77 of the center rod 76 is closed manually, and the center rod 76 is pushed into the fixing member 73. The bone members 75 lose support from the large diameter part 76b of the fixing member 73, and change from the spread state to the closed state. At the same time, the internal rail 78 is also pushed by the bone members 75, and an axis of the internal rail 78 matches that of the center rod 76. In this state, the center rod 76 is pulled out from the opening 2a of the body wall 2, with the fixing member 73.

According to the endoscope apparatus 102 as described above, the internal insertion portion 1 can be moved along the internal rail 78, which extends in any directions, and can be turned. Therefore, it is possible to move the internal insertion portion 1 relative to the body wall 2 without restricting the position of the opening 2a made in the body wall 2, and it is possible to observe the target area from any direction. Further, since the internal insertion portion 1 is coupled with the operation unit 3 so that the body wall 2 is interposed between the internal insertion portion 1 and the operation unit 3, it is easy to intuitively understand the position from which the image displayed on the monitor 8 is captured, and it is easy to perform orientation. As a result, it is possible to treat the target area appropriately. Those advantages are the same as those of the first embodiment.

Additionally, in the third embodiment, the internal insertion portion 1 is moved inside of the body cavity by the internal rail 78 and the center rod 76, which can be turned, in other words, the internal insertion portion 1 is moved in the vicinity of the polar coordinate. Therefore, it is possible to move the internal insertion portion 1 smoothly. Further, since the bone members 75 are spread inside of the body cavity, it is unnecessary to perform aeroperitoneum by generally using CO₂ gas. As a result, it is possible to provide the operation space inside of the body cavity.

### Fourth Embodiment

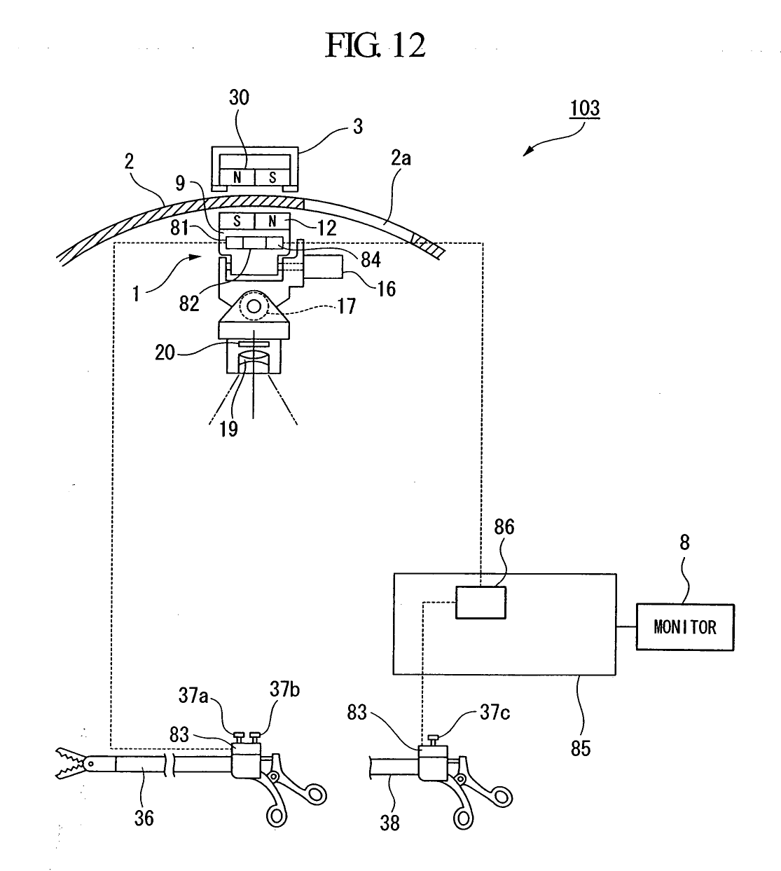

An explanation of an endoscope apparatus 103 relating to the fourth embodiment of the present invention is described hereafter, with reference to FIG. 12.

FIG. 12 is a view showing a state where the part of the endoscope apparatus has been inserted into the body cavity.

This fourth embodiment differs from the above-mentioned first embodiment as follows. That is, the endoscope apparatus 103 does not include the guide portion 22, and the operation unit 3 is directly moved on the body wall 2. Further, the internal insertion portion 1 is provided with a receiving portion 81 and a driving processor 82, and the rigid forceps 36, 38 are provided with a transmitting portion 83. Furthermore, the internal insertion portion 1 is provided with a transmitting portion 84, and an image processor 85 which is disposed outside of the body is provided with a receiving portion 86.

That is, for example, the receiving portion 81, the driving processor 82 and receiving portion 84 are built in the base portion 9 of the internal insertion portion 1. The receiving portion 81 receives a signal sent from the transmitting portion 83 of the rigid forceps 36. Further, an image signal captured by the imaging element 20 is subjected to a predetermined process, and is sent to the receiving portion 86 of the image processor 85. The receiving portion 86 of the image processor 85 also receives a signal which is sent from the transmitting portion 83 connected to the zoom switch 37c of the rigid forceps 38.

Next, an explanation of the operation of the above composed endoscope apparatus 103 is provided.

The internal insertion portion 1 is inserted inside of the body cavity through the opening 2a made in the body wall 2, and the operation unit 3 is arranged outside of the body wall 2. Thus, the internal insertion portion 1 is attached to the inner surface of the body wall 2 corresponding to the operation unit 3 by the magnetic force 12, 30. Then, the operation unit 3 is held manually, and the operation unit 3 is moved along the body wall 2 in the back and forth directions, and in the right and left directions. Thus, it is possible to synchronously move the internal insertion portion 1 to an optional position inside of the body cavity, by the magnetic force 12, 30.

When the movement is finished, the operation unit 3 is released. However, the internal insertion portion 1 is fixed on the position by an interposing force which acts between the internal insertion portion 1 and the operation unit 3 by the magnetic force 12, 30.

Image zooming and angle changing are performed as follows. That is, by the switches 37a, 37b which are attached to the rigid forceps 36 being operated, a signal is sent to the receiving portion 81 disposed inside of the internal insertion portion 1 through the transmitting portion 83 by wireless, thus the motors 16, 17 are controlled through the driving processor 82.

Similarly, an image inside of the body cavity which is captured by the internal insertion portion 1 is sent to the receiving portion 86 of the image processor 85 through the transmitting portion 84 disposed inside of the internal insertion portion 1, then is displayed by the monitor 8. When it is required to enlarge the image, by the switch 37c which is attached to the rigid forceps 38 being operated, a signal is sent to the receiving portion 86 of the image processor 85 through the transmitting portion 83 by wireless. An image signal is subjected to a predetermined process, and an enlarged image is displayed by the monitor 8.

According to the endoscope apparatus 103 as described above, the internal insertion portion 1 can be supported by the operation unit 3 using an attaching force of the magnets 12, 30 so as to interpose the body wall 2 therewith. Therefore, it is possible to move the internal insertion portion 1 relative to the body wall 2 without restriction of the position of the opening 2a made in the body wall 2, and it is possible to observe the target area from any direction. Further, since the internal insertion portion 1 is coupled with the operation unit 3 so that the body wall 2 is interposed between the internal insertion portion 1 and the operation unit 3, it is easy to intuitively understand the position from which the image displayed on the monitor 8 is captured, and it is easy to perform orientation. As a result, it is possible to treat the target area appropriately. Those advantages are the same as those of the first embodiment.

Additionally, in the fourth embodiment, there is no guide portion 22 which guides the operation unit 3 such as the rectilinear rail and the arched rail, accordingly it is possible to downsize the device, and to reduce costs. In addition, it is possible to easily prepare setting the device.

While preferred embodiments of the invention have been described and illustrated above, it should be understood that these are exemplary of the invention and are not to be considered as limiting. Modifications can be made without departing from the spirit or scope of the present invention.

For example, in the first and fourth embodiments, for attaching the operation unit 3 and the internal insertion portion to each other, the magnets 12, 30 are respectively disposed on the operation unit 3 and the internal insertion portion so as to face each other. However, one of the magnet 12 or the magnet 30 may be replaced with a magnetic body.

In the embodiments as described above, the imaging element 20 is built in the internal insertion portion 1. However, the imaging element 20 may be replaced with a treating unit which performs predetermined treatment relative to the target area, or the internal insertion portion 1 may be provided with the treating unit with the imaging element 20.

In the present embodiment, it is possible to synchronously move the operation unit 3 and the internal insertion portion 1 by the magnetic force. However, the magnetic force may be used only for fixing the internal insertion portion 1 to an inner surface of the body wall, and the internal insertion portion 1 may be unmovably fixed inside of the body cavity.

In this case, the object optical system 19 of the internal insertion portion 1 is provided with a wide-angle optical system which is able to capture the entire area of the body cavity in sight, and the monitor 8 displays only one part of an image which is captured. Also, an area which is desired to be displayed by the monitor 8 can be moved in the back and forth directions, and in the right and left directions using an inputting device secured to the operation unit 3, and the area can be specified. Therefore, any desirable area can be displayed by the monitor 8 among the entire image area of the object optical system 19. By doing this, it is possible to change the view.

In this case, the internal insertion portion 1 is not only fixed to the inner surface of the body wall by the magnetic force, but the internal insertion portion 1 is also fixed to the inner surface of the body wall by a different mechanical device.

Further, it is conceivable to secure the view by the internal insertion portion 1 being moved freely, without fixing the internal insertion portion 1 to the inner surface of the body wall.

For example, liquid such as water or saline is filled in the body cavity, and the internal insertion portion 1 is arranged in the liquid. Then, the internal insertion portion 1 can be moved in the liquid by a signal which is sent from outside of the body. Note that, a buoyant force acts on the internal insertion portion 1 arranged in the liquid. When the liquid is moved, a reaction force is caused, and the internal insertion portion 1 can be moved by the reaction force. Therefore, it is advantageous that the internal insertion portion 1 is arranged in the liquid.

In detail, the internal insertion portion 1 is provided with a device for obtaining thrust such as a propeller, and the device may be controlled by wireless outside of the body. Further, by balancing the magnetic force acting from outside of the body cavity with the weight of the internal insertion portion 1, the distance between the internal insertion portion 1 and the target area can be adjusted. Furthermore, by moving the magnetic force generator, a magnetic field outside of the body cavity changes. Therefore, it is possible to move the internal insertion portion 1.

What is claimed is:

1. An endoscope apparatus comprising:
an endoscope which has an imaging element; and
a monitor which displays an image imaged by the imaging element; wherein
the endoscope is provided with an internal insertion portion which is movably arrangeable inside a body cavity, and an operation unit for changing the position of the internal insertion portion, which is arrangeable outside the body, and wherein
the internal insertion portion and the operation unit are arranged on a common axis so as to interpose a body wall therebetween.

2. The endoscope apparatus according to claim 1, further comprising a coupler which moves the internal insertion portion following the movement of the operation unit while the operation unit is guided along the body wall by a guide portion.

3. The endoscope apparatus according to claim 2, wherein the operation unit is moved along the guide portion manually.

4. The endoscope apparatus according to claim 2, wherein the operation unit is moved along the guide portion by the driving force of a motor.

5. The endoscope apparatus according to claim 4, wherein a rigid forceps separately provided from the endoscope, a tip end of the rigid forceps being inserted into the body cavity, and the rigid forceps are provided with an inputting unit for operating the motor.

6. The endoscope apparatus according to claim 2, wherein the coupler is composed of a magnet which is disposed on one of either the body wall side end portion of the internal insertion portion or the body wall side end portion of the operation unit, and a magnet or a magnetic body which is disposed on the other of the body wall side end portion of the internal insertion portion or the body wall side end portion of the operation unit, the magnet or the magnetic body pulling the magnet.

7. The endoscope apparatus according to claim 2, wherein the coupler comprises plural elongated bodies which connect the internal insertion portion and the operation unit, and an elongated body length adjustment unit which reels up or reels out the elongated bodies.

8. The endoscope apparatus according to claim 2, wherein the guide portion comprises two rectilinear rails which are disposed parallel to each other, and an arched rail which is disposed between the two rectilinear rails and is movable along a longitudinal direction of the two rectilinear rails, and wherein
the arched rail movably guides the operation unit along an arched path thereof.

9. The endoscope apparatus according to claim 2, further comprising a intracoelomic guide portion which is inserted into the body cavity through an opening, and which guides the internal insertion portion.

10. The endoscope apparatus according to claim 2, wherein the internal insertion portion comprises a base portion which is moved in the body cavity following the movement of the operation unit by the coupler, and an observation portion main body which is attached to the base portion through a joint portion so as to be a variable angle with respect to the base portion and which is attached to the imaging element.

11. The endoscope apparatus according to claim 1, wherein the internal insertion portion is provided with a treating unit which performs a predetermined treatment in the body cavity.

12. An endoscope apparatus comprising:
an observation unit or a treating unit which is movably arrangeable inside a body cavity;
an operation unit of the observation unit or the treating unit which is arranged on an optical axis of the observation unit or an operating axis of the operating unit, and which is movably arrangeable outside the body;
a magnet which is disposed on one of either the body wall side end portion of the observation unit or the treating unit or the body wall side end portion of the operation unit; and
a magnet or a magnetic body which is disposed on the other of either the body wall side end portion of the observation unit or the treating unit or the body wall side end portion of the operation unit, and which pulls the magnet.

13. An endoscope apparatus comprising:
an observation unit or a treating unit which is movably arrangeable inside a body cavity;
an operation unit of the observation unit or the treating unit which is arranged on an optical axis of the observation unit or an operating axis of the operation unit, and which is movably arrangeable outside the body;
an elongated body which is connected between the observation unit or the treating unit and the operation unit through an opening disposed in a body wall;
an amount of movement measuring unit which measures an amount of movement of the operating unit relative to the body wall; and
an elongated body reel up unit which reels up the elongated body by twice the length of the amount of movement of the operating unit measured by the amount of movement measuring unit, when the operating unit is moved a predetermined distance in the direction in which the elongated body is slackened.
